(19) United States
(12) Patent Application Publication
(10) Pub. No.: US 2023/0277430 A1
(43) Pub. Date: Sep. 7, 2023

(54) COSMETIC PRODUCT WITH HIGH DEGREE OF EMPTYING OF THE PREPARATION FROM THE PACKAGING

(71) Applicant: BEIERSDORF AG, Hamburg (DE)

(72) Inventors: Claudia MUELLER, Tangstedt (DE); Katrin RUPP, Hamburg (DE); Svea WISCHHOEFER, Hamburg (DE); Katharina HERWIG, Hamburg (DE); Andreas BLECKMANN, Ahrensburg (DE)

(21) Appl. No.: 18/000,965

(22) PCT Filed: May 21, 2021
(86) PCT No.: PCT/EP2021/063713

§ 371 Date: Dec. 7, 2022

(30) Foreign Application Priority Data
Jun. 10, 2020 (DE) 10 2020 003 488.8

(51) Int. Cl. A61K 8/73; A61Q 19/00

(52) U.S. Cl. CPC A61K 8/732; A61Q 19/00; A61K 2800/87; A61K 2800/30

(57) ABSTRACT

Cosmetic product of a) a cosmetic preparation containing hydroxypropyl starch phosphate (INCI: Hydroxypropyl Starch Phosphate) and b) a packaging with a dispensing opening in the form of a hole from which the cosmetic preparation can be dispensed in the form of a flow.

The present invention relates to a cosmetic product composed of a cosmetic preparation comprising hydroxypropyl starch phosphate (INCI: Hydroxypropyl Starch Phosphate) and packaging having a dispensing opening in the form of a hole from which the cosmetic preparation can be dispensed in the form of a flow, and to a process and to use of hydroxypropyl starch phosphate (INCI: Hydroxypropyl Starch Phosphate) in packaging having a dispensing opening in the form of a hole from which the cosmetic preparation can be dispensed in the form of a flow.

The desire to look beautiful and attractive is naturally rooted in humans. Although ideals of beauty have changed over time, the pursuit of a flawless appearance has always been aimed for by humans. An essential part of a beautiful and attractive appearance is the condition and complexion of the skin.

In order for the skin to be able to perform the full range of its biological functions, it requires regular cleansing and care. Skin care products, generally creams, ointments or lotions, mostly serve for moisturizing and refatting the skin. Active ingredients are commonly added thereto, which are intended to regenerate the skin and for example to prevent and reduce the premature aging thereof (e.g. the appearance of fine lines and wrinkles).

In addition to cleansing and caring for the skin, cosmetics also have an aesthetic function. They are intended to “improve” the external appearance of the user according to the respective cultural ideas. Cosmetics thus fulfil a psychological and social function, since they increase the (visual) attractiveness of the user. This area includes above all “decorative” cosmetics, which change the appearance of the user with the help of colorants applied to the skin. Indirectly, however, cleansing and care products also have a positive influence, since clean, healthy skin corresponds to people's ideal of beauty.

The use of known ingredients such as mineral oils, silicone oils and polyacrylates is practically always at the expense of disadvantages with respect to the application of the products. The preparations become sensorially unattractive, which is unpleasantly noticeable, for example, when spreading the preparation on the skin and the lack of absorption capacity. Thus, inter alia, replacing mineral oils, silicone oils and polyacrylates such as carbomers, acrylates/C10-30 alkyl acrylate crosspolymers, results in increased “whitening” when the preparation is rubbed on the skin.

Therefore, the object of the present invention was to find a replacement for mineral oils and silicone oils in cosmetic preparations, which has the same sensory properties (particularly with regard to whitening, the spreadability on the skin and the ability to be absorbed) as the mineral and silicone oils.

Closely linked to the trend towards “natural” cosmetics is the consumer's desire to consume cosmetics in a resource-saving manner. In addition to recyclable packaging materials, as complete a removal as possible of the cosmetic preparation is part of sustainable use. However, according to the prior art, it is very difficult to completely remove highly viscous preparations (such as lotions and creams) from tubes and bottles. Quite often a not inconsiderable amount remains in the packaging.

It was therefore the object of the present invention to develop a cosmetic product (i.e., a cosmetic preparation in a packaging material) in which the so-called degree of emptying of residues (i.e., the removal of the preparation from the packaging material as completely as possible) is significantly increased compared to the prior art.

The objects are achieved by a cosmetic product of
a) a cosmetic preparation comprising hydroxypropyl starch phosphate (INCI: Hydroxypropyl Starch Phosphate) and
b) packaging having a dispensing opening in the form of a hole from which the cosmetic preparation can be dispensed in the form of a flow.

The objects are also achieved by using hydroxypropyl starch phosphate (INCI: Hydroxypropyl Starch Phosphate) in cosmetic preparations for increasing the degree of emptying residues of the preparation from packaging having a dispensing opening in the form of a hole from which the cosmetic preparation can be dispensed in the form of a flow.

Last but not least, the objects are achieved by a process for producing a cosmetic product with high degree of emptying residues, characterized in that
a) hydroxypropyl starch phosphate (INCI: Hydroxypropyl Starch Phosphate) is added to the preparation and
b) packaging is selected having a dispensing opening in the form of a hole from which the cosmetic preparation can be dispensed in the form of a flow.

In the context of the present disclosure, the phrases “according to the invention”, “advantageous according to the invention” etc. always refer to the cosmetic product according to the invention, the use according to the invention and the process according to the invention, unless otherwise described in the individual case.

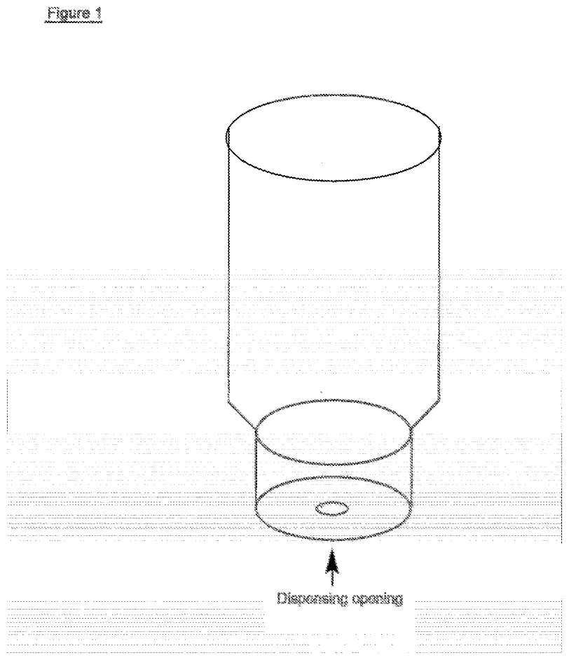

According to the invention, it is advantageous if the walls of the packaging have a gradient towards the dispensing opening. This can be seen in FIG. 1 by way of example. The gradient towards the dispensing opening ensures that the preparation (for example under the influence of gravity) can completely slip off the walls towards the opening (sliding down) without being prevented from doing so by geometric obstructions.

Embodiments of the present invention that are advantageous according to the invention are characterized in that the preparation comprises hydroxypropyl starch phosphate (INCI: Hydroxypropyl Starch Phosphate) at a concentration of 0.2 to 5% by weight, based on the total weight of the preparation. In this case, a content from 0.5 to 2.5% by weight, based on the total weight of the preparation, is preferred in accordance with the invention.

Particularly advantageous are such hydroxypropyl starch phosphates as described in U.S. Pat. No. 6,248,338. The use of a hydroxypropyl distarch phosphate, such as that sold as Structure® XL by Nouryon, is especially preferred.

It is advantageous in the context of the present invention if the preparation is free from polyacrylates and carbomers.

In addition, it is advantageous in the context of the present invention if the preparation is free from 3-(4-methylbenzylidene)camphor, 2-hydroxy-4-methoxybenzophenone (INCI: Oxybenzone), 2-ethylhexyl 4-methoxycinnamate (INCI Octyl Methoxycinnamate), 2-ethylhexyl 2-cyano-3,3-diphenylacrylate, homomenthyl salicylate, parabens (particularly propyl and butyl paraben), methylisothiazolinone, chloromethylisothiazolinone and DMDM hydantoin, polyethylene glycol ethers or polyethylene glycol esters and is free from mineral oils, silicone oils and mineral waxes.

Rather, it is advantageous according to the invention if the preparation comprises one or more oils selected from the group of compounds comprising Butylene Glycol Dicaprylate/Dicaprate, Phenethyl Benzoate, C12-15 Alkyl Benzoate, Dibutyl Adipate; Diisopropyl Sebacate, Dicaprylyl Carbonate, Di-C12-13 Alkyl Tartrate, Butyloctyl Salicylate, Diethylhexyl Syringylidenemalonate, Hydrogenated Castor Oil Dimerate, Triheptanoin, C12-13 Alkyl Lactate, C16-17 Alkyl Benzoate, Propylheptyl Caprylate, Diethylhexyl 2,6-Naphthalate, Octyldodecanol, Caprylic/Capric Triglyceride, Ethylhexyl Cocoate, Cococaprylate/Caprate, Cocoglycerides, Dicaprylyl Ether.

According to the invention, preference is given to the use of Cococaprylate/Caprate, Cocoglycerides, Dicaprylyl Ether.

If the preparation comprises Cococaprylate/Caprate, this component is advantageously used according to the invention at a concentration of 2 to 6% by weight, based on the total weight of the preparation.

If the preparation contains Cocoglycerides, this component is advantageously used according to the invention at a concentration of 2 to 6% by weight, based on the total weight of the preparation.

If the preparation contains Dicaprylyl Ether, this component is advantageously used according to the invention at a concentration of 1 to 4% by weight, based on the total weight of the preparation.

The preparation according to the invention is advantageously present in accordance with the invention in the form of an emulsion. Oil-in-water emulsions (O/W emulsions) are preferred in accordance with the invention.

In such a case, it is advantageous according to the invention if the preparation comprises one or more O/W emulsifier(s) selected from the group of the compounds glyceryl stearate citrate, glyceryl stearate (self-emulsifying), stearic acid, stearate salts, polyglyceryl-3 methylglucose distearate, sodium cetearyl sulfate, potassium cetyl phosphate, polyglyceryl-10 stearate, sodium stearoyl glutamate. These can be used at the usual application concentrations.

With regard to the packaging, it is advantageous according to the invention if the packaging contains HDPE. According to the invention, the packaging preferably consists of HDPE.

Advantageous embodiments of the present invention are characterized in that the preparation comprises stearyl alcohol and/or cetyl alcohol. According to the invention, these fatty alcohols can advantageously be used at a total concentration of 0.5 to 8% by weight, based on the total weight of the preparation, with this concentration specification also applying to cases in which only one of the fatty alcohols is present in the preparation.

It is advantageous in the context of the present invention if the preparation comprises one or more alkanediols from the group of the compounds pentane-1,2-diol, hexane-1,2-diol, octane-1,2-diol, decane-1,2-diol, 2-methylpropane-1,3-diol, ethylhexylglycerin and/or 4-hydroxyacetophenone.

Furthermore, advantageous embodiments according to the invention are obtained in that the preparation contains cetyl palmitate and/or xantham gum.

If the preparation contains cetyl palmitate, this substance is advantageously used according to the invention at a concentration of 0.5 to 5% by weight, based on the total weight of the preparation.

If the preparation contains xanthan gum, this substance is advantageously used according to the invention at a concentration of 0.5 to 5% by weight, based on the total weight of the preparation.

It is advantageous in accordance with the invention if the preparation comprises ethanol. It is preferable in accordance with the invention to use ethanol at a concentration of 3 to 8% by weight based on the total weight of the preparation.

Embodiments of the present invention that are advantageous in accordance with the invention are characterized in that the preparation contains one or more compounds selected from the group of the compounds alpha-lipoic acid, folic acid, phytoene, D-biotin, coenzyme Q10, alpha-glucosylrutin, carnitine, carnosine, natural and/or synthetic isoflavonoids, flavonoids, creatine, creatinine, taurine, β-alanine, panthenol, magnolol, honokiol, tocopheryl acetate, dihydroxyacetone, 8-hexadecene-1,16-dicarboxylic acid, thiamidol, glycerylglucose, (2-hydroxyethyl)urea, vitamin E and derivatives thereof, hyaluronic acid and/or salts thereof, and/or licochalcone A.

The preparation according to the invention may advantageously contain moisturizers. Moisturizers are substances or mixtures of substances that give cosmetic preparations the property, when applied or spread on the surface of the skin, of reducing the loss of moisture from the stratum corneum (also known as transepidermal water loss (TEWL)) and/or of having a beneficial effect on the hydration of the stratum corneum.

Advantageous moisturizers according to the present invention are, for example, glycerin, lactic acid and/or lactates, in particular sodium lactate, butylene glycol, propylene glycol, biosaccharide gum-1, glycine soya, ethylhexyloxyglycerin, pyrrolidone carboxylic acid and urea. Furthermore, it is particularly advantageous to use polymeric moisturizers from the group of polysaccharides which are soluble in water and/or swellable in water and/or gellable with the aid of water. Particularly advantageous, for example, are hyaluronic acid, chitosan and/or a fucose-rich polysaccharide, which is obtainable in Chemical Abstracts under the name Fucogel® 1000 from SOLABIA S.A. It is also possible to use moisturizers advantageously as anti-wrinkle agents, for protection against skin changes which occur during ageing of the skin for example.

The cosmetic preparations according to the invention may also advantageously comprise fillers, although not mandatory, which further improve, for example, the sensory and cosmetic properties of the formulations and which generate or enhance, for example, a velvety or silky skin feel. Advantageous fillers in the context of the present invention are starch and starch derivatives (such as tapioca starch, distarch phosphate, aluminum or sodium starch octenyl succinate and the like), pigments, which have principally neither UV filter nor staining effects (e.g., boron nitride etc.) and/or Aerosils® (CAS No. 7631-86-9) and/or talc.

It is preferred in accordance with the invention if the preparation according to the invention comprises tapioca starch and/or distarch phosphate.

The water phase of the preparations according to the invention may advantageously contain customary cosmetic auxiliaries, such as alcohols, in particular those having a low number of carbon atoms, preferably ethanol and/or isopropanol or polyols of low carbon number and ethers thereof, preferably propylene glycol, glycerin, electrolytes, self-tanners, etc.

It is further advantageous in the context of the invention if the preparation according to the invention contains one or more perfumes selected from the group of the compounds limonene, citral, linalool, alpha-isomethyl ionone, geraniol, citronellol, 2-isobutyl-4-hydroxy-4-methyltetrahydropyran, 2-tert-pentylcyclohexyl acetate, 3-methyl-5-phenyl-1-pentanol, 7-acetyl-1,1,3,4,4,6-hexamethyltetralin, adipic acid diesters, alpha-amylcinnamaldehyde, alpha-methyl ionone, amyl C butylphenylmethylpropionalcinnamal, amyl salicylate, amylcinnamyl alcohol, anise alcohol, benzoin, benzyl alcohol, benzyl benzoate, benzyl cinnamate, benzyl salicylate, bergamot oil, bitter orange oil, butylphenyl methylpropional, cardamom oil, cedrol, cinnamal, cinnamyl alcohol, citronellyl methylcrotonate, lemon oil, coumarin, diethyl succinate, ethyl linalool, eugenol, Evernia furfuracea extract, Evernia prunastri extract, farnesol, guaiac wood oil, hexylcinnamal, hexyl salicylate, hydroxycitronellal, lavender oil, limonene oil, linayl acetate, mandarin oil, menthyl PCA, methylheptenone, nutmeg oil, rosemary oil, sweet orange oil, terpineol, tonka bean oil, triethyl citrate and/or vanillin.

It is advantageous according to the invention if the preparation has a viscosity of 1500 to 8500 mPas, measured using a viscometer of the Rheomat 123 type from Pro Rheo using measuring body 1.

Embodiments of the present invention that are advantageous according to the invention are further characterized in that they have a yield point of 9 to 23 Pa.

In the context of the present invention, the critical shear stress of the flow curve is considered to be the yield point.

The flow curve is measured at 25° C.+/−1° C. on a shear stress-controlled rheometer SR-2000 type from Rheometric Scientific with 25 mm plate/plate geometry with a gap between 0.8 mm and 1.2 mm, with structure-preserving filling. A suitable constant shear stress time ramp is predefined and an appropriate structural recovery time is observed before the test and the critical shear stress at the maximum of the yield curve is specified.

### COMPARATIVE TEST/WORKING EXAMPLE

The examples below are intended to illustrate the present invention without limiting it. Unless otherwise stated, all quantitative data, fractions, and percentages are based on the weight and the total amount or on the total weight of the preparations.

| INCI | Comparative test 1 | Comparative test 2 | Comparative test 3 |
|---|---|---|---|
| Coco-glycerides | 3 | 0 | 3 |
| Cetyl Palmitate | 0.5 | 0.5 | 0.5 |
| Coco-Caprylate/Caprate | 3 | 0 | 3 |
| Dicaprylyl Ether | 1.5 | 0 | 1.5 |
| Paraffinum Liquidum | 0 | 7.5 | 0 |
| Glyceryl stearate SE | 2 | 2 | 2 |
| Glyceryl Stearate | 0.5 | 0.5 | 0.5 |
| Sodium cetearyl sulfate | 0.15 | 0.15 | 0.15 |
| Glycerin | 7 | 7 | 7 |
| Alcohol Denat. + Aqua | 5 | 5 | 5 |
| Cetearyl alcohol | 4 | 4 | 4 |
| Carbomer | 0 | 0 | 0.1 |
| Xanthan Gum | 0.1 | 0.1 | 0 |
| Hydroxypropyl Starch | 0.75 | 0.75 | 0 |
| Phosphate + Aqua | | | |
| Preservative | qs | qs | qs |
| Aqua + Sodium Hydroxide | 0 | 0 | Addition to pH 6.5-7.5 |
| Citric Acid | Addition to pH 6-7 | Addition to pH 6-7 | 0 |
| Perfume | qs | qs | qs |
| Aqua | to 100 | to 100 | to 100 |

Procedure

The tare weight of the packaging is first determined.

The packaging material composed of PE in the form of a lotion bottle with a possible capacity of ca. 250 ml is filled with 200 ml of the preparation.

The packaging material composed of PE in the form of a lotion bottle (see FIGURE for geometry), with a possible capacity of ca. 400 ml, is filled with 350 ml of the preparation.

The weight of the filled packaging is determined.

Now the preparation is squeezed out of the bottle by opening the cap, turning the bottle upside down with the opening downwards, and squeezing it together with the hands until no more preparation can be removed from the bottle. These samples are then stored upside down for several hours (4-24 hours) with the cap closed. Thereafter, another attempt is made to remove the preparation from the bottle by opening the cap and squeezing the bottle together with the hands until no more preparation can be removed from the bottle. Optionally, to facilitate removal, the bottle is vertically shaken vigorously several times with the cap closed. This procedure is repeated until no more preparation can actually be removed.

The weight of the empty packaging is determined and the residual amount of preparation left in the bottle is calculated.

Dimensions of the 200 ml lotion bottle:
Height: 17 cm
Width: 6.3 cm

Dimensions of the 350 ml lotion bottle:
Height: 20 cm
Width: 7.3 cm

Order Numbers of the Packaging:

Bottle (Alpla Noblejas):
200 ml—65952-90025-00
350 ml—25649-20025-00

Caps (Berry Spain):
Cap—65953-90025-00

Test Result of Residual Emptying

| | Comparative test 1 | Comparative test 2 | |
|---|---|---|---|
| | Hydroxypropyl | Hydroxypropyl | Comparative test 3 |
| | starch phosphate | starch phosphate | Carbomer |
| Tare weight of the 200 ml | 14.3 | 14.3 | 14.3 |
| packaging in g | | | |
| Weight of the filled 200 ml | 217.7 | 187.6 | 204.3 |
| packaging in g | | | |
| Filled preparation in g | 203.4 | 173.3 | 190 |
| Weight of the emptied 200 ml | 26.9 | 20.6 | 33.6 |
| packaging in g | | | |
| Residual content of the | 12.6 | 6.3 | 19.3 |
| preparation in the 200 ml | | | |
| packaging in g | | | |
| Residual amount in % | 6.19 | 3.64 | 10.16 |
| Tare weight of the 350 ml | 20.5 | 21 | 20.3 |
| packaging in g | | | |
| Weight of the filled 350 ml | 369.3 | 321.7 | 348.3 |
| packaging in g | | | |
| Filled preparation in g | 348.8 | 300.7 | 328 |
| Weight of the emptied 350 ml | 28.6 | 27.9 | 40.7 |
| packaging in g | | | |
| Residual content of the | 8.1 | 6.9 | 20.4 |
| preparation in the 350 ml | | | |
| packaging in g | | | |
| Residual amount in % | 2.32 | 2.29 | 6.22 |

1.-15. (canceled)

16. A cosmetic product, wherein the product comprises
(a) a cosmetic preparation comprising hydroxypropyl starch phosphate and
(b) a packaging having a dispensing opening in the form of a hole from which the cosmetic preparation can be dispensed in the form of a flow.

17. The cosmetic product of claim 16, wherein walls of the packaging have a gradient towards the dispensing opening.

18. The cosmetic product of claim 16, wherein the preparation comprises from 0.2% to 5% by weight of hydroxypropyl starch phosphate, based on a total weight of the preparation.

19. The cosmetic product of claim 18, wherein the preparation comprises from 0.5% to 2.5% by weight of hydroxypropyl starch phosphate.

20. The cosmetic product of claim 16, wherein the preparation is free from polyacrylates and carbomers.

21. The cosmetic product of claim 16, wherein the preparation is free from 3-(4-methylbenzylidene)camphor, 2-hydroxy-4-methoxybenzophenone, 2-ethylhexyl 4-methoxycinnamate, 2-ethylhexyl 2-cyano-3,3-diphenylacrylate, homomenthyl salicylate, parabens, methylisothiazolinone, chloromethylisothiazolinone, DMDM hydantoin, polyethylene glycol ethers, polyethylene glycol esters, mineral oils, silicone oils, and mineral waxes.

22. The cosmetic product of claim 16, wherein the preparation further comprises one or more oils selected from Butylene Glycol Dicaprylate/Dicaprate, Phenethyl Benzoate, C12-15 Alkyl Benzoate, Dibutyl Adipate; Diisopropyl Sebacate, Dicaprylyl Carbonate, Di-C12-13 Alkyl Tartrate, Butyloctyl Salicylate, Diethylhexyl Syringylidenemalonate, Hydrogenated Castor Oil Dimerate, Triheptanoin, C12-13 Alkyl Lactate, C16-17 Alkyl Benzoate, Propylheptyl Caprylate, Diethylhexyl 2,6-Naphthalate, Octyldodecanol, Caprylic/Capric Triglyceride, Ethylhexyl Cocoate, Cocoglycerides, Dicaprylyl Ether.

23. The cosmetic product of claim 16, wherein the preparation is present as an O/W emulsion.

24. The cosmetic product of claim 16, wherein the preparation further comprises one or more O/W emulsifier(s) selected from glyceryl stearate citrate, glyceryl stearate (self-emulsifying), stearic acid, stearate salts, polyglyceryl-3 methylglucose distearate, sodium cetearyl sulfate, potassium cetyl phosphate, polyglyceryl-10 stearate, sodium stearoyl glutamate.

25. The cosmetic product of claim 16, wherein the packaging comprises HDPE.

26. The cosmetic product of claim 16, wherein the preparation comprises stearyl alcohol and/or cetyl alcohol.

27. The cosmetic product of claim 16, wherein the preparation further comprises one or more contains one or more of pentane-1,2-diol, hexane-1,2-diol, octane-1,2-diol, decane-1,2-diol, 2-methylpropane-1,3-diol, ethylhexylglycerin, 4-hydroxyacetophenone.

28. The cosmetic product of claim 16, wherein the preparation further comprises cetyl palmitate and/or xanthan gum.

29. The cosmetic product of claim 16, wherein the preparation further comprises ethanol.

30. The cosmetic product of claim 16, wherein the preparation has a viscosity of from 1800 mPas to 8500 mPas, as measured using a viscometer Rheomat 123 from proRheo using measuring body 1.

31. The cosmetic product of claim 16, wherein the preparation has a yield point of from 9 Pa to 23 Pa.

32. A method of increasing the degree of emptying residues of a preparation from a packaging, wherein the method comprises using a preparation which comprises hydroxypropyl starch phosphate and a packaging having a dispensing opening in the form of a hole from which the preparation can be dispensed in the form of a flow.

33. The method of claim 32, wherein the preparation comprises from 0.2% to 5% by weight of hydroxypropyl starch phosphate, based on a total weight of the preparation.

34. The method of claim 32, wherein walls of the packaging have a gradient towards the dispensing opening.

35. A process for producing a cosmetic product exhibiting a high degree of emptying of residues, wherein the process comprises using a cosmetic preparation which comprises hydroxypropyl starch phosphate and a packaging having a dispensing opening in the form of a hole from which the preparation can be dispensed in the form of a flow.
